(19) United States
(12) Patent Application Publication
(10) Pub. No.: US 2021/0187079 A1
(43) Pub. Date: Jun. 24, 2021

(54) ORAL OCTREOTIDE FOR THE TREATMENT OF DISEASE

(71) Applicant: Chiasma, Inc., Waltham, MA (US)

(72) Inventors: Roni Mamluk, Mazkeret Batya (IL); Gary Patou, Los Altos Hills, CA (US); Dana Gelbaum, Tel Aviv (IL); Asi Haviv, Gan-Shlomo (IL)

(21) Appl. No.: 16/071,630

(22) PCT Filed: Jan. 20, 2017
(86) PCT No.: PCT/US2017/014379

§ 371 Date: Jul. 20, 2018

Related U.S. Application Data
(60) Provisional application No. 62/303,072, filed on Mar. 3, 2016.
(60) Provisional application No. 62/299,607, filed on Feb. 25, 2016.
(60) Provisional application No. 62/281,320, filed on Jan. 21, 2016.

(51) Int. Cl. A61K 38/31; A61P 1/16; A61P 1/12; A61P 13/12; A61K 9/00; A61K 45/06

(52) U.S. Cl. CPC A61K 38/31; A61P 1/16; A61K 45/06; A61P 13/12; A61K 9/0053; A61P 1/12

(57) ABSTRACT

The present invention relates to oral therapy of a subject suffering from disease e.g. polycystic disease e.g. polycystic kidney disease or polycystic liver disease or PCOS or hypotension especially neurogenic orthostatic hypotension and postprandial hypotension, or intractable diarrhea or neuroendocrine tumors or carcinoid syndrome. The method of treatment comprises oral administration to the subject of a therapeutically effective amount of an oral somatostatin receptor ligand (SRL) e.g. oral octreotide. Combination therapy of oral SRLs with other drugs is also envisaged, e.g. octreotide in combination with a therapeutically effective amount of a second therapeutic agent and optionally in combination with a therapeutically effective amount of a third therapeutic agent.

### CLAIM OF PRIORITY

This application claims priority to U.S. Ser. No. 62/281,320, filed Jan. 21, 2016; U.S. Ser. No. 62/299,607, filed Feb. 25, 2016; and, U.S. Ser. No. 62/303,072, filed Mar. 3, 2016; the contents of each of which is incorporated herein by reference.

### FIELD OF THE TECHNOLOGY

The present invention relates to oral delivery of octreotide alone and in combination with other therapeutic agents for the treatment of various diseases including polycystic disease (polycystic kidney disease, polycystic liver disease, polycystic ovarian syndrome), hypotension especially neurogenic orthostatic hypotension and postprandial hypotension, intractable diarrhea of various types, neuroendocrine tumors and carcinoid syndrome.

### BACKGROUND

The use in clinical trials of injectable octreotide or lanreotide—which are somatostatin receptor ligands (SRLs)—for the treatment of various diseases e.g. polycystic liver disease and polycystic kidney disease is described in the literature. See Lariviere et al 2015, Translational Research, 165 (4), 488-498, Elsevier Inc; Chapman et al (2015) Kidney International, 17-27; Chandok (2012) Annals of Hepatology, 11 (6), 819-826; and Cnossen and Drenth (2014) Orphanet Journal of Rare Diseases, 9, 69. Neuroendocrine tumors and carcinoid syndrome are described in the literature e.g. Kloppel et al 2004, Ann. N.Y. Acad Sci, 1014:13-17. Combination treatment of injectable octreotide or lanreotide with other therapeutic agents for treatment of neuroendocrine tumors and carcinoid syndrome is described in the literature. Examples are Oberg et al 2004, Annals of Oncology 15:966-973; Oberg et al 2012, Annals of Oncology 23 (Supplement 7): vii124-vii130; Strosberg et al (2017) New England J Med, 376:2, 125-135; and Pavel et al (2016) Clin. Pharmacol and Therapeutics advance online publication doi:10.1002/cpt.559. Injectable octreotide has been used for intractable/refractory diarrhea and this has been reviewed by Szilagyi and Shrier (2001) Aliment Pharmacol Ther. 15:1889-1897. Treatment of secretory diarrhea in general has been reviewed by Thiagarajah and Donowitz (2015) August; 12(8):446-57. Guidelines for treatment of cancer-treatment induced diarrhea have been described by Benson et al (2004) J. of Clin. Oncology, 22 (14), 2918-2926. Injectable octreotide has been used for management of short bowel syndrome and this has been described by Nehra et al (2001) Am. J. of Gastroenterology, 96(5), 1484-1498; treatment of short bowel syndrome has been discussed by Parrish et el (March 2015) Practical Gastroenterology, 28-42. Injectable octreotide has been used for management of dumping syndrome and this has been described by Didden et al (2006) Aliment Pharmacol Ther, 24: 1367-1375. Injectable octreotide has been used for management of diarrhea in HIV-infected individuals and this has been described by MacArthur and DuPont (2012) Clinical Infectious Diseases, 55(6):860-867.

There is a need for use of an oral somatostatin receptor ligand (SRL) e.g. oral octreotide alone and in combination treatment with other therapeutic agents for the treatment of various diseases including polycystic disease (for example polycystic kidney disease, polycystic liver disease, polycystic ovarian syndrome), hypotension especially neurogenic orthostatic hypotension and postprandial hypotension, intractable diarrhea of various types, neuroendocrine tumors and carcinoid syndrome. Particular advantages of oral administration are avoidance of often painful injections, avoidance of injection site reactions and reduction in breakthrough symptoms.

### SUMMARY

The present invention relates to therapy of a subject suffering from polycystic disease (for example polycystic kidney disease and/or polycystic liver disease or other diseases such as polycystic ovarian syndrome), hypotension especially neurogenic orthostatic hypotension and postprandial hypotension, intractable diarrhea of various types, neuroendocrine tumors and carcinoid syndrome. The method of treatment comprises administration to the subject of a therapeutically effective amount of an oral somatostatin receptor ligand (SRL) e.g. oral octreotide.

Additionally, the present invention relates to combination therapy of a subject suffering from these diseases. One method of treatment comprises administration to the subject of a therapeutically effective amount of an oral somatostatin receptor ligand (SRL) e.g. oral octreotide in combination with a therapeutically effective amount of a second and optionally a third therapeutic agent.

For example, one method of treatment comprises administration to the subject of a therapeutically effective amount of an oral somatostatin receptor ligand (SRL) e.g. oral octreotide in combination with a therapeutically effective amount of an angiotensin-converting enzyme inhibitor e.g. lisinopril, administered orally. Another method of treatment comprises administration to the subject of a therapeutically effective amount of an oral somatostatin receptor ligand (SRL) e.g. oral octreotide in combination with a therapeutically effective amount of an angiotensin receptor blocker (also termed angiotensin II receptor antagonist) e.g. telmisartan (Micardis™) administered orally. Another method of treatment comprises administration to the subject of a therapeutically effective amount of an oral somatostatin receptor ligand (SRL) e.g. oral octreotide in combination with a therapeutically effective amount of an arginine vasopressin V2 receptor antagonist e.g. tolvaptan, administered orally. Another method of treatment comprises administration to the subject of a therapeutically effective amount of an oral somatostatin receptor ligand (SRL) e.g. oral octreotide in combination with a therapeutically effective amount of a statin e.g. pravastatin administered orally. Another method of treatment comprises administration to the subject of a therapeutically effective amount of an oral somatostatin receptor ligand (SRL) e.g. oral octreotide in combination with a therapeutically effective amount of an Src kinase inhibitor (also termed a tyrosine kinase inhibitor) e.g. bosutinib, administered orally; bosutinib is marketed under the trade name Bosulif®. Another method of treatment comprises administration to the subject of a therapeutically effective amount of an oral somatostatin receptor ligand (SRL) e.g. oral octreotide in combination with a therapeutically effective amount of an mTOR inhibitor e.g. everolimus, administered orally (Afinitor® of Novartis) or sirolimus, also termed rapamycin (Rapamune® of Pfizer) administered orally. Another method of treatment comprises administration to the subject of a therapeutically effective amount of an oral somatostatin receptor ligand (SRL) e.g. oral octreotide in combination with a therapeutically effective amount of a drug which treats diarrhea e.g. loperamide, cholestyramine, atropine or an opioid (e.g. codeine, diphenoxyate or difenoxin). Another method of treatment comprises administration to the subject of a therapeutically effective amount of an oral somatostatin receptor ligand (SRL) e.g. oral octreotide in combination with a therapeutically effective amount of teduglutide or L-glutamine.

Oral octreotide may also be administered in combination with injectable octreotide to control breakthrough symptoms.

Another aspect of this invention is a unit dosage formulation for oral administration comprising a therapeutically effective amount of octreotide and a therapeutically effective amount of an angiotensin-converting enzyme inhibitor; in a particular aspect the angiotensin-converting enzyme inhibitor is lisinopril. Another aspect of this invention is a unit dosage formulation for oral administration comprising a therapeutically effective amount of octreotide and a therapeutically effective amount of an angiotensin receptor blocker; in a particular aspect the angiotensin receptor blocker is telmisartan. Another aspect of this invention is a unit dosage formulation for oral administration comprising a therapeutically effective amount of octreotide and a therapeutically effective amount of an arginine vasopressin V2 receptor antagonist; in a particular aspect the arginine vasopressin V2 receptor antagonist is tolvaptan. Another aspect of this invention is a unit dosage formulation for oral administration comprising a therapeutically effective amount of octreotide and therapeutically effective amount of a statin; in a particular aspect the statin is pravastatin. Another aspect of this invention is a unit dosage formulation for oral administration comprising octreotide and a Src kinase inhibitor; in a particular aspect the Src kinase inhibitor is bosutinib. Another aspect of this invention is a unit dosage formulation for oral administration comprising a therapeutically effective amount of octreotide and a a therapeutically effective amount of mTOR inhibitor; in a particular aspect the mTOR inhibitor is everolimus or sirolimus (also termed rapamycin). Additionally, the unit dosage formulation for oral administration may include a therapeutically effective amount of octreotide plus a therapeutically effective amount of two or more additional drugs selected from an angiotensin-converting enzyme inhibitor, an angiotensin receptor blocker, an arginine vasopressin V2 receptor antagonist, statin, Src kinase inhibitor and mTOR inhibitor. Additionally, a unit dosage formulation for oral administration may include a therapeutically effective amount of octreotide plus a therapeutically effective amount of one or more additional drugs selected from loperamide, cholestyramine, atropine, an opioid (e.g. codeine, diphenoxylate or difenoxin), teduglutide, L-glutamine and telotristat etiprate.

The invention also relates to a method of treatment of a subject suffering from a neuroendocrine tumor which comprises administration to the subject of a therapeutically effective amount of an oral somatostatin receptor ligand (SRL) in combination with a therapeutically effective amount of one or more anti-tumor agents or an mTOR inhibitor or an VEGFR inhibitor or an Src kinase inhibitor or a tryptophan hydroxylase inhibitor or an injectable somatostatin receptor ligand (SRL) or telotristat etiprate.

Throughout this application, various publications, including United States patents, are referenced by author and year and patents and applications by number. The disclosures of these publications and patents and patent applications in their entireties are hereby incorporated by reference into this application in order to more fully describe the state of the art to which this invention pertains.

### DETAILED DESCRIPTION

### Polycystic Disease

Polycystic kidney disease (PKD or PCKD, also known as polycystic kidney syndrome) is a genetic disorder in which abnormal cysts develop and grow in the kidneys. Cystic disorders can express themselves at any point, infancy, childhood, or adulthood. The disease occurs in humans and some animals. PKD is characterized by the presence of multiple cysts (hence, “polycystic”) typically in both kidneys; however, 17% of cases initially present with observable disease in one kidney, with most cases progressing to bilateral disease in adulthood. Polycystic kidney disease is a general term for the two types of PKD, each having their own pathology and causes. The two types of PKD are autosomal dominant polycystic kidney disease (ADPKD) and autosomal recessive polycystic kidney disease (ARPKD), which differ in their mode of genetic inheritance.

Autosomal dominant polycystic kidney disease (ADPKD) is the most common of all the inherited cystic kidney diseases with an incidence of 1:500 live births. Studies show that 10% of end-stage kidney disease (ESKD) patients being treated with dialysis in Europe and the U.S. were initially diagnosed and treated for ADPKD.

Mutations in the PKD-1 and PKD-2 genes account for the overwhelming majority of ADPKD cases. Fewer than 10% of cases of ADPKD appear in non-ADPKD families. Cyst formation begins in utero from any point along the nephron, although fewer than 5% of nephrons are thought to be involved. As the cysts accumulate fluid, they enlarge, separate entirely from the nephron, compress the neighboring kidney parenchyma, and progressively compromise kidney function, typically leading to kidney failure by the sixth decade of life. Kidneys can enlarge to 3 to 4 times their normal size.

Autosomal recessive polycystic kidney disease (ARPKD) is the less common of the two types of PKD, with an incidence of 1:20,000 live births and is typically identified in the first few weeks after birth. Unfortunately, the kidneys are often underdeveloped resulting in a 30% death rate in newborns with ARPKD.

Currently there are no Food and Drug Administration-approved treatments for polycystic kidney disease. Several drugs designed to slow or arrest the progression of polycystic kidney disease have shown promise in preclinical or in clinical trials. These include injectable SRLs and other drugs. A significant barrier to the development of an effective therapy for polycystic kidney disease has been the lack of a means to measure the progression of the disease. Serum creatinine is not usually increased until late in the course of the disease by which time significant and irreversible damage to the renal parenchyma has already occurred. Measurement of total kidney volume (TKV) in relation to age can identify a patient with progressive disease. TKV is an accurate estimate of kidney cyst burden as associated with pain, hypertension, gross hematuria, proteinuria or albuminuria, and loss of kidney function. TLV increases exponentially in virtually every ADPKD patient with an average of 5-6% per year in adults. Elevated TKV, particularly when used together with age and kidney function, identifies individuals who are at risk for progression to end stage renal disease (ESRD). See Lariviere et al 2015, Translational Research, 165 (4) 488-498, Elsevier Inc; and Chapman et al (2015) Kidney International, 17-27.

The US Food and Drug Administration currently accepts halving of glomerular filtration rate (GFR), assessed as doubling of serum creatinine level, as a surrogate end point for the development of kidney failure in clinical trials of kidney disease progression. A doubling of serum creatinine level generally is a late event in chronic kidney disease (CKD); thus, there is great interest in considering alternative end points for clinical trials to shorten their duration, reduce sample size, and extend their conduct to patients with earlier stages of CKD. However, the relationship between lesser declines in GFR and the subsequent development of kidney failure has not been well characterized. The National Kidney Foundation and Food and Drug Administration sponsored a scientific workshop in 2012 to examine critically available data to determine whether alternative GFR-based end points have sufficiently strong relationships with important clinical outcomes of CKD to be used in clinical trials. Based on a series of meta-analyses of cohorts and clinical trials and simulations of trial designs and analytic methods, the workshop concluded that a confirmed decline in estimated GFR of 30% over 2 to 3 years may be an acceptable surrogate end point in some circumstances, but the pattern of treatment effects on GFR must be examined, specifically acute effects on estimated GFR. An estimated GFR decline of 40% may be more broadly acceptable than a 30% decline across a wider range of baseline GFRs and patterns of treatment effects on GFR.

Polycystic liver disease comes in two forms as ADPKD (with kidney cysts) and ADPLD (liver cysts only). Liver cysts occur in more than 80% of adults with ADPKD. In the vast majority of patients, the liver cysts are asymptomatic but they can grow uncomfortably large and cause pain. Unlike the kidney failure that inevitably results from polycystic kidney disease, PLD does not normally lead to liver failure and, in fact, most patients do not require surgery. In a minority of patients, polycystic liver disease creates a myriad of symptoms from the compressive effect of enlarged cysts and can even cause malnutrition and liver decompensation in the severest of cases. In patients with symptomatic disease a variety of interventional radiology or surgical techniques can be considered, including aspiration with sclerotherapy of a dominant cyst, fenestration, segmental hepatic resection and even liver transplantation.

Currently there are no Food and Drug Administration—approved treatments for polycystic liver disease. Several drugs designed to slow or arrest the progression of polycystic liver disease have shown promise in preclinical or in clinical trials. These include injectable SRLs and other drugs. Total liver volume (TLV), total kidney volume (TKV) and changes in glomerular filtration rate may be measured. See Chandok (2012) Annals of Hepatology, 11(6), 819-826; and Cnossen and Drenth (2014) Orphanet Journal of Rare Diseases, 9, 69.

Polycystic ovary syndrome (PCOS), one of the most common causes of ovulatory infertility, affects 4-7% of women. PCOS may have some genetic component and clinical features of this disorder may change throughout a life span, starting from adolescence to postmenopausal age. In young women with PCOS, hyperandrogenism, menses irregularities, and insulin resistance may occur together, emphasizing the pathophysiological role of excess androgen and insulin on PCOS. Hyperandrogenism and infertility represent the major complaints of PCOS in adult fertile age. In addition, obesity and metabolic syndrome may affect more than half these women. Later in life, it becomes clear that the association of obesity (particularly the abdominal phenotype) and PCOS renders affected women more susceptible to develop type 2 diabetes mellitus (T2DM), with some difference in the prevalence rates among countries, suggesting that environmental factors are important in determining individual susceptibility. Little is known about ovarian morphology and androgen production in women with PCOS after menopause. Some studies found that morphological ultrasonographic features consistent with polycystic ovaries are very common in postmenopausal women, and that these features are associated with higher than normal testosterone levels and metabolic alterations. (Pasquali 2006 Annals of the New York Academy of Sciences 1092(1):158-74. January 2007.)

Currently, there are no FDA-approved drugs that treat PCOS; treatments today are used off-label to treat one, or multiple, categories of PCOS-associated symptoms: uteroprotection, hyperandrogenism, fertility and metabolic health.

### Hypotension

Neurogenic Orthostatic Hypotension (nOH) is a subtype of orthostatic hypotension that occurs in people with an existing neurologic disease (e.g., neurological conditions that are chronic and irreversible). In some embodiments, the neurologic disease is Parkinson's Disease or Multi-System Atrophy (MSA). Orthostatic (postural) hypotension refers to a reduction in systolic blood pressure (e.g., of at least 20 mm Hg) or a reduction in diastolic blood pressure (e.g., of at least 10 mm Hg) during the first 3 minutes of standing. Neurogenic orthostatic hypotension can be caused by autonomic nervous system malfunction, which is the part of the nervous system controlling involuntary body activity (e.g., keeping blood pressure normal). Symptoms include dizziness, light-headedness, syncope (fainting), fatigue, blurry vision, weakness, trouble concentrating, head and neck pain. Outcomes include injuries such as tooth damage, broken bones, even death as a result of falling.

Postprandial hypotension is commonly defined as a decrease in systolic blood pressure of 20 mmHg or more observed within two hours after meal ingestion. It is very common in older patients especially in those living in long-term healthcare homes. Patients with postprandial hypotension may develop symptomatic hypotension, syncope (fainting) and falls. See Lisk, R. (April 2010) Postprandial hypotension in www.gerimed.co.uk, Cardiology 203-206; and Lubart et al (September 2006) Journal of the American Geriatrics Society, Vol. 54, Issue 9, pages 1377-1381, Postprandial Hypotension in Long-Term Care Elderly Patients on Enteral Feeding

### Diarrhea

Diarrheal disease remains a major health burden worldwide. Secretory diarrheas are caused by certain bacterial and viral infections, inflammatory processes, drugs and genetic disorders. Fluid secretion across the intestinal epithelium in secretory diarrheas involves multiple ion and solute transporters, as well as activation of cyclic nucleotide and Ca2+ signalling pathways. Current treatment of diarrhea includes replacement of fluid and electrolyte losses using oral rehydration solutions, and drugs targeting intestinal motility or fluid secretion.

Diarrhea may have many causes and may be intractable (also termed refractory). It may be due to dumping syndrome, or to short bowel syndrome, or may be caused by chemotherapy, by radiotherapy, by HIV/AIDS or by a neuroendocrine tumor (e.g. a carcinoid tumor or a Vasoactive Intestinal Peptide (VIP) secreting adenoma) or due to graft-versus-host disease, irritable bowel syndrome (IBS), inflammatory bowel disease (which includes conditions that cause the gut to become inflamed, such as Crohn's disease and ulcerative colitis), coeliac disease (also termed celiac sprue), chronic pancreatitis, diverticular disease, endocrine disorders, vasculitis, post-surgical diarrhea, carbohydrate malabsorption syndrome, amyloidosis, lactose intolerance, small bowel bacterial overgrowth, hepatobiliary disorders, inadequate luminal bile acid, bile acid malabsorption, loss of regulated gastric emptying, pancreatic exocrine insufficiency or neoplasia e.g. bowel cancer or may be due to be due to invasive infectious disease and/or bacterial endotoxins e.g. cholera. See Juckett, G. (2011) Evaluation of chronic diarrhea. Am Fam Physician. 84(10).

Octreotide exerts pharmacologic actions similar to the natural hormone, somatostatin. Like somatostatin, it is a potent inhibitor of growth hormone, glucagon, insulin, and inhibits release of serotonin, gastrin, vasoactive intestinal peptide, secretin, motilin, and pancreatic polypeptide. It also suppresses LH response to GnRH and decreases splanchnic blood flow.

By virtue of these pharmacological actions, octreotide has been used to treat symptoms associated with metastatic carcinoid tumors (flushing and diarrhea), and Vasoactive Intestinal Peptide (VIP) secreting adenomas (watery diarrhea). Octreotide may be used to treat flushing and/or diarrhea associated with other diseases as described herein. See Szilagyi et el (2002) Aliment. Parmacol. Ther. 15, 1889-1897.

Dumping syndrome occurs when food, especially sugar, moves too fast from the stomach to the duodenum—the first part of the small intestine—in the upper gastrointestinal (GI) tract. This condition is also called rapid gastric emptying. Dumping syndrome has two forms, based on when symptoms occur early dumping syndrome, which occurs 10 to 30 minutes after a meal and late dumping syndrome, which occurs 2 to 3 hours after a meal. Dumping syndrome is caused by problems with the storage of food particles in the stomach and emptying of particles into the duodenum. Early dumping syndrome results from rapid movement of fluid into the intestine following a sudden addition of a large amount of food from the stomach. Late dumping syndrome results from rapid movement of sugar into the intestine, which raises the body's blood glucose level and causes the pancreas to increase its release of the hormone insulin. The increased release of insulin causes a rapid drop in blood glucose levels (hypoglycemia). People who have had surgery to remove or bypass a significant part of the stomach are more likely to develop dumping syndrome. Some types of gastric surgery, such as bariatric surgery, reduce the size of the stomach. As a result, dietary nutrients pass quickly into the small intestine. Other conditions that impair how the stomach stores and empties itself of food, such as nerve damage caused by esophageal surgery, can also cause dumping syndrome.

Short bowel syndrome (also termed SBS or short gut syndrome or simply short gut) is a malabsorption disorder caused by the surgical removal of the small intestine for the treatment of GI conditions (e.g., severe Crohn's disease, traumatic injury, cancer) or more rarely due to the complete dysfunction of a large segment of bowel due to diseases that directly disrupt small intestine nutrient absorption (e.g., chronic intestinal pseudo-obstruction syndrome, refractory sprue).

SBS patients suffer from impaired nutrient absorption that may lead to malnutrition, diarrhea, cramping, bloating, heartburn, weakness, and fatigue. Symptom severity is dependent on the length and functionality of remaining bowel. Nutrient deficiencies may be specific to the section of the bowel that is removed. SBS significantly impacts the patient's quality of life and it is expensive to manage given potential requirements for supplemental nutrition. Parenteral nutrition requires 8-12-hour IV infusions.

Most cases of SBS are acquired, although some children are born with a congenital short bowel. SBS usually does not develop unless more than two thirds of the small intestine has been removed. Physicians segment SBS patients for management based on the length of remaining functional intestine, as follows:
Mild: patients with minimal functional loss who maintain the ability to obtain nutrition solely through enteral feeding (includes both oral and tube feeding).
Moderate: patients require intermittent parenteral nutrition 1-4 days per week to supplement enteral feeding.
Severe: patients are incapable of enteral feeding and require total parenteral nutrition i.e. infusions 5-7 days per week.

Current drug treatment of short bowel syndrome can include teduglutide (Gattex®) and/or L-glutamine and/or somatropin (human growth hormone). It can also include histamine2-receptor agonists (H2 blockers), proton pump inhibitors (PPIs) and clonidine. See Byrne et al (1995) J. of Parenteral and Enteral medicine, 19 (4): 296-302; Jeppesen et al (2005) Gut 54:1224-1231 and Parrish et el (March 2015) Practical Gastroenterology, 28-42.

Chemotherapy-induced diarrhea (CID) and radiation-induced diarrhea occur as a result of various types of gastrointestinal insults and injuries that are associated with prolonged treatment. Diarrheal conditions may be a consequence of the toxic effect of chemotherapeutics and/or radiotherapy on the gastrointestinal tract, or an inflammatory condition caused by damaged and/or modified gut flora. Gastrointestinal complications due to chemotherapy and radiotherapy are largely inflammatory by nature and include: panenteritis, enterocolitis, mucositis (broadly defined inflammation within the bowels and small intestine); abdominal pain (localized pain in the gastrointestinal system); autoimmune colitis (autoimmune bowel disease characterized by inflammation); ischemic colitis (inflammation of bowel as a result of inadequate blood supply); and gastrointestinal leukocytoclastic vasculitis (inflammation of bowel due to small-vessel vasculitis).

Chemotherapy-induced diarrhea and radiation-induced diarrhea are characterized as a side-effect of pelvic or abdominal radiotherapy and as a side-effect of wide variety of chemotherapeutics, including antimetabolites, plant alkaloids, cytotoxic antibodies, and alkylating agents. Chemotherapy-induced diarrhea is most commonly caused by fluoropyrimidines, particularly fluorouracil (FU), capecitabine and irinotecan.

HIV/AIDS-induced diarrhea includes diarrhea as a secondary manifestation of HIV infection, immunodeficiency, HIV-related enteropathy or medication side effects.

### Neuroendocrine Tumors (NET)

Thousands of patients have neuroendocrine tumors that originate in the gastrointestinal tract and metastasize or spread to the liver or other organs. Overproduction of serotonin within these metastatic neuroendocrine tumor (mNET) cells is a driver of carcinoid syndrome, which is characterized by debilitating diarrhea, facial flushing, abdominal pain, heart valve damage, and other serious consequences. Thus carcinoid syndrome is a subset of neuroendocrine tumors that have specific symptomatic manifestations, due to secretion of vasoactive substances into the systemic circulation.

The severe and unpredictable diarrhea associated with carcinoid syndrome has a profound impact on the lives of cancer patients, often preventing them from participating in daily activities. Patients with carcinoid syndrome can live for many years with metastatic cancer, requiring the need for long-term treatment options to effectively manage their disease

The current standard of care for carcinoid syndrome is somatostatin analog depot injection (SSA), first approved in 1998. SSA is also termed somatostatin receptor ligand (SRL). SRL injection therapy fails over time to maintain adequate control of carcinoid syndrome for most patients, with many becoming not adequately controlled within the first two years after the therapy is initiated. This decrease in response to a drug after its administration is termed tachyphylaxis.

There are three main types of neuroendocrine tumors, classified by origin of the tumor endocrine cells—pancreatic neuroendocrine tumors, gastrointestinal neuroendocrine tumors and pulmonary tumors. A pancreatic neuroendocrine tumor which secretes vasoactive intestinal peptide (VIP) is called VIPoma.

Neuroendocrine tumors and carcinoid syndrome and the medical treatments employed in the treatment of these conditions are described in the literature eg Kloppel et al 2004, Ann. N.Y. Acad Sci 1014:13-17; Strosberg (2014) Endocr. Prac. 20(2); 167-175; Oberg et al 2004, Annals of Oncology 15:966-973; Oberg et al 2012, Annals of Oncology 23 (Supplement 7): vii124-vii130; Schmidt et al (2011) Oncogene, 30:1497-1505.

Drug treatment of NET: Somatostatin receptor ligand (SRL) therapy remains the backbone of therapy for patients with NET, and patients are generally treated with long-acting octreotide; injectable lanreotide has also been recently used.

If metastasis of carcinoid tumor has occurred and in cases where surgical excision is not suitable, NET may treated be treated with currently recommended chemotherapy.

Anti tumor agents currently used or in clinical trials to treat or palliate NET include the following: alkylating agents, doxorubicin, fluoropyrimidines e.g. 5-fluorouracil, dacarbazine, actinomycin D, platinum compounds (cisplatin, carboplatin, oxaliplatin), irinotecan, etoposide, streptozotocin (STZ), interferon alfa, interferon gamma, bortezomib (iv/sc) marketed as Velcade®, temozolomide (oral) marketed as Temodar®), bevacizumab, capecitabine and somatostatin analogs with a radioactive load (e.g. octreotide attached to a radioactive load using for example yttrium-90 or 111 indium-labeling agents; one example is Lutathera®. Lutathera (lutetium Lu 177 dotatate) is a Lu-177-labeled somatostatin analog peptide currently in development for the treatment of gastroenteropancreatic neuroendocrine tumors (GEP-NETs), including foregut, midgut, and hindgut neuroendocrine tumors in adults; see Strosberg et al (2017) New England J Med, 376:2, 125-135. Anti-tumor agents may be chemotherapeutic agents or radiotherapeutic agents. A combination of these chemotherapeutic/anti-tumor agents is typically used e.g. cisplatin/etoposide or streptozotocin/5-fluorouracil/doxorubicin or capecitabine/bevacizumab or temozolomide/capecitabine.

Other therapeutic agents which may be used to treat NET are as follows: an mTOR inhibitor such as everolimus (oral) marketed as Afinitor®), temsirolimus (intravenous) marketed as Torisel® and sirolimus (oral) marketed as Rapamune®; an oral VEGFR inhibitor such as sunitinib (marketed as Sutent®); an Src kinase inhibitor (also termed a tyrosine kinase inhibitor) e.g. bosutinib, administered orally, marketed as Bosulif®; and a tryptophan hydroxylase inhibitor e.g. telotristat etiprate also termed LX1032, administered orally. Sunitinib (sunitinib malate) is a targeted tyrosine kinase inhibitor able to inhibit members of the receptor tyrosine kinases families containing a split-kinase domain; these families include VEGF receptor (VEGFR) types 1, 2 and 3 and other receptors. Telotristat etiprate is the first investigational drug in clinical studies to target tryptophan hydroxylase, an enzyme that triggers the excess serotonin production within mNET cells that leads to carcinoid syndrome

Chemoembolization of the hepatic artery for treatment of metastatic carcinoid tumor has been widely used in adults for treatment of NET. Other treatments, which may also be considered as required, include liver-directed therapy, such as radiofrequency ablation, radioembolization, chemoembolization, and rarely surgical debulking.

Breakthrough NET symptoms (due to hormonal symptoms associated with NETs) are common phenomena in patients receiving injectable octreotide e.g. octreotide LAR (long-acting formulation). See for example Dasari, November 2014 Oncology Initial Treatment of Well-Differentiated Neuroendocrine Tumors. Patients may require short-acting octreotide in addition to octreotide LAR, typically 100-250 μg up to 3 times per day for breakthrough symptoms, especially for the first 10 to 14 days after LAR injection while awaiting therapeutic levels. In patients with progressive or poorly controlled symptoms, somatostatin analog doses may be increased as needed. These additional daily s.c. injections may be effective in controlling the breakthrough symptoms, yet significantly increase the physical, emotional, and financial burden of the treatment.

The current invention includes the treatment of patient suffering from NET or any of the diseases described herein by treatment with an oral SRL e.g. octreotide, optionally in combination with one or more other therapeutic agents. Another aspect of the invention is the use of oral octreotide administered in addition to long-acting SRLs or other therapies to prevent or treat breakthrough symptoms. This “rescue therapy” may be given on a regular basis towards the beginning or end of the four-week dosing interval or on an “on demand basis” when symptoms such as diarrhea, facial flushing or abdominal pain occur. The breakthrough symptoms (e.g. in the case of carcinoid syndrome or small bowel syndrome) may be an increase in number or volume of stools.

The current invention includes the treatment of NET by treatment with an oral SRL e.g. oral octreotide in combination with one or more other therapeutic agents. Options include (a) oral SRL in combination with one or more of the anti-tumor (chemotherapeutic or radiotherapeutic) agents as listed above; (b) oral SRL in combination with an mTOR inhibitor e.g. everolimus, temsirolimus or sirolimus (c) oral SRL in combination with a VEGFR inhibitor e.g. sunitinib; (d) oral SRL in combination with a Src kinase inhibitor e.g. bosutinib; (e) a tryptophan hydroxylase inhibitor (also known as a serotonin synthesis inhibitor) e.g. telotristat etiprate; and (f) oral SRL in combination with an injectable SRL.

The oral SRL may be oral octreotide, lanreotide or pasireotide or an oral formulation of DG3173; DG3173 is also termed somatoprim, and is a novel SRL, administered as subcutaneous bolus injections.

One measure of the success of the treatment, comprising oral octreotide in combination with a second or third therapeutic agent, is a reduction in the average number of daily bowel movements of the subject suffering from NET after some weeks of treatment e.g. 6-12 weeks compared with baseline.

Another measure of the success of the treatment, comprising oral octreotide in combination with a second or third therapeutic agent, is a reduction in the volume of daily bowel movements of the subject suffering from NET after some weeks of treatment e.g. 6-12 weeks compared with baseline.

Yet another measure of the success of the treatment, comprising oral octreotide in combination with a second or third therapeutic agent, is a reduction in the average number of flushing episodes of the subject suffering from NET after some weeks of treatment e.g. 6-12 weeks compared with baseline.

Another measure of the success of the treatment, comprising oral octreotide in combination with a second or third therapeutic agent is improvement of progression-free survival of the subject suffering from NET.

The current invention includes the treatment of patient suffering from any of the diseases described herein by oral treatment with an oral SRL e.g. octreotide, optionally in combination with one or more other therapeutic agents

In some embodiments, the tablet or capsule comprising octreotide is about 10 to about 30 mg (e.g., about 15 to about 25 mg, about 18 to about 22 mg, about 20 mg) octreotide.

Another aspect of this invention is a unit dosage formulation for oral administration comprising octreotide and a second oral therapeutic agent for treating NET; in a particular aspect the second oral drug is a anti-tumor (radiotherapeutic or chemotherapeutic) agent, a MTOR inhibitor, an oral VEGFR inhibitor, an Src kinase inhibitor, or a tryptophan hydroxylase inhibitor or a combination thereof.

In particular aspects of this invention the administration of oral octreotide comprises about 5 mg to about 400 mg of octreotide daily, about 40 to about 300 mg of octreotide daily, or about 10 to about 200 mg of octreotide daily, such as 10, 20, 30, 40, 50, 60, 70, 80 or 100 or 200 or 300 or 400 or more mg daily. A particular dosage of oral octreotide is 80 mg daily. The daily dose of octreotide may be divided into one or two doses a day e.g. the 80 mg daily dose may be administered in two doses of 40 mg each.

### Somatostatin Receptor Ligands (SRLs)

Injectable SRLs as currently used may be octreotide (e.g. Sandostatin® and Sandostatin® LAR), lanreotide which is a cyclic octapeptide (eg Somatuline® Depot in the US and Somatuline® Autogel elsewhere), pasireotide, DG3173 or CAM2029. Pasireotide is a cyclic hexapeptide and is also known as Signifor® or SOM-230; DG3173 is also termed somatoprim, and is a novel SRL, administered as subcutaneous bolus injection; and CAM2029 is a subcutaneous depot injection with octreotide as active ingredient.

SRLs may be given in a “long-acting” formulation (e.g. depot formulation or other slow release formulation) or in a “short-acting” (e.g. immediate release) formulation. Sandostatin® is a short-acting formulation administered subcutaneously (sc) and Sandostatin® LAR is a long-acting formulation administered by intramuscular (im) injection. Somatuline® Depot is a long-acting formulation and Signifor® is a short-acting formulation which may be administered subcutaneously once or twice a day or more.

The “short-acting” formulation is normally a subcutaneous injection given daily (or even two or three times a day or more), or may be given 2, 3, 4, 5, or 6 times per week. The “long-acting” formulation is normally given by means of injection at dosing intervals of four weeks, or alternatively at dosing intervals of 3-8 weeks e.g. at 3, 4, 5, 6, 7, or 8 weeks. The interval between two injections of long-acting SRLs is termed the dosing interval.

The oral SRL used in the methods of the instant invention may be oral octreotide, oral lanreotide or oral pasireotide or an oral formulation of DG3173. Oral formulations of octreotide have been described and claimed, for example in co-assigned U.S. Pat. No. 8,329,198 which is hereby incorporated by reference; see for example claims 1-26. The oral octreotide may be in a capsule or a tablet. One aspect of the current invention, which has novel and useful benefits, is an oral formulation of octreotide, in combination with one or more other therapeutic agents as described herein.

Octreotide is a cyclic octapeptide (e.g. a salt such as acetate or chloride) and is an analog (agonist) of the natural hormone somatostatin; it mimics somatostatin pharmacologically, though it is a more potent inhibitor of growth hormone, glucagon and insulin than the natural hormone. The molecular weight of octreotide is 1019.3 (free peptide, C49H66N10O10S2).

### Oral Dosage Forms

In an embodiment, the oral octreotide is administered in an oral dosage form described herein. An exemplary oral dosage forms includes an enteric-coated oral dosage form. This may comprise a composition comprising a suspension which comprises an admixture of a hydrophobic medium and a solid form wherein the solid form comprises a therapeutically effective amount of octreotide, at least one salt of a medium chain fatty acid and polyvinylpyrrolidone (PVP), wherein the polyvinylpyrrolidone is present in the composition at an amount of 2% or more by weight (e.g., about 2% to about 20% by weight or about 5% to about 15% by weight), and wherein the at least one salt of a medium chain fatty acid salt is present in the composition at an amount of at least 10% or more by weight (e.g., about 10% to 40% by weight or about 12% to 18% by weight). In some embodiments, the solid form further includes one or more excipients.

In some embodiments of the compositions described herein, the solid form including the therapeutic agent also includes a stabilizer (e.g., a stabilizer of protein structure). Stabilizers of protein structure are compounds that stabilize protein structure under aqueous or non-aqueous conditions or can reduce or prevent aggregation of the therapeutic agent, for example during a drying process such as lyophilization or spray-drying or other processing step. Stabilizers of structure can be polyanionic molecules, such as phytic acid, polyvalent ions such as Ca, Zn or Mg, saccharides such as a disaccharide (e.g., trehalose, maltose) or an oligo or polysaccharide such as dextrin or dextran, or a sugar alcohol such as mannitol, or an amino acid such as glycine, or polycationic molecules, such as spermine, or surfactants such as Tween 80 or Span 40 or pluronic acid. Uncharged polymers, such as methyl cellulose and polyvinyl alcohol, are also suitable stabilizers.

In an embodiment, the hydrophobic medium comprises glyceryl tricaprylate and the solid form consists of polyvinylpyrrolidone with a molecular weight of about 3000, and sodium octanoate. In an embodiment, the hydrophobic medium additionally comprises castor oil or glyceryl monocaprylate or a combination thereof and a surfactant. In an embodiment, the hydrophobic medium consists of glyceryl tricaprylate, glyceryl monocaprylate, and polyoxyethylene sorbitan monooleate.

In an embodiment, the solid form consists essentially of octreotide, polyvinylpyrrolidone with a molecular weight of about 3000, and sodium octanoate. In an embodiment, the composition comprises about 41% of glyceryl tricaprylate, about 27% castor oil, about 4% glyceryl monocaprylate, about 2% polyoxyethylene sorbitan monooleate, about 15% sodium octanoate, about 10% polyvinylpyrrolidone with a molecular weight of about 3000, less than 1% water and octreotide. In an embodiment, the composition comprises about 68% glyceryl tricaprylate, about 4% glyceryl monocaprylate, about 2% polyoxyethylene sorbitan monooleate, about 15% sodium octanoate, about 10% polyvinylpyrrolidone with a molecular weight of about 3000, about 1% water and a therapeutically effective amount of octreotide. In an embodiment, the composition comprises a therapeutically effective amount of octreotide, about 12-21% of sodium octanoate, about 5-10% of polyvinylpyrrolidone with a molecular weight of about 3000, about 20-80% of glyceryl tricaprylate, about 0-50% castor oil, and about 3-10% surfactant. In an embodiment, the composition comprises a therapeutically effective amount of octreotide, about 12-21% of sodium octanoate, about 5-10% of polyvinylpyrrolidone with a molecular weight of about 3000, about 20-80% of glyceryl tricaprylate, and about 3-10% surfactant.

In an embodiment, the octreotide is present at an amount of less than 33% (e.g., less than 25%, less than 10%, less than 1%, or less than 0.1%). In an embodiment, the composition comprises about 15% of sodium octanoate, about 10% of polyvinylpyrrolidone with a molecular weight of about 3000, about 30-70% glyceryl tricaprylate and about 6% of surfactant. In an embodiment, the surfactant is glyceryl monocaprylate or polyoxyethylene sorbitan monooleate.

In an embodiment, the solid form comprises a particle or a plurality of particles. In an embodiment, the solid form further comprises a stabilizer.

In an embodiment, the polyvinylpyrrolidone has a molecular weight of about 3000.

In an embodiment, the medium chain fatty acid salt has a chain length from about 6 to about 14 carbon atoms. In an embodiment, the medium chain fatty acid salt is sodium hexanoate, sodium heptanoate, sodium octanoate, sodium nonanoate, sodium decanoate, sodium undecanoate, sodium dodecanoate, sodium tridecanoate or sodium tetradecanoate, or a corresponding potassium or lithium or ammonium salt or a combination thereof. In an embodiment, the medium chain fatty acid salt is sodium octanoate.

In an embodiment, the hydrophobic oily medium comprises a mineral oil, a paraffin, a fatty acid a monoglyceride, a diglyceride, a triglyceride, an ether or an ester, or a combination thereof. In an embodiment, the medium chain fatty acid salt is a lithium, potassium or ammonium salt. In an embodiment, the hydrophobic oily medium comprises glyceryl tricaprylate. In an embodiment, the composition further comprises a surfactant.

In some embodiments, the pharmaceutical composition includes a plurality of therapeutic agents i.e. octreotide and one (or more) additional therapeutic agents. The therapeutic agents can either be in the same solid form (e.g., in the same particle), or the therapeutic agents can each be in an independent solid form (e.g., each in different particles. In some embodiments, the therapeutic agent is in the form of a particle, for example, a granulated or solid particle. The particle is associated with or is in intimate contact with a substantially hydrophobic medium, for example, a hydrophobic medium described herein.

In general, the composition may include from about 1.0% to about 30% by weight of the therapeutic agent e.g. about 1, 2, 3, 4, 5, 6, 7, 8, 9, 10, 15, 20, 25 or 30% by weight. The maximum by weight of the therapeutic agent included in the composition is often in the range of about 5%-20%.

Oral octreotide for clinical trials is provided as an enteric-coated capsule containing 20 mg of octreotide (20 mg calculated as free base), polyvinylpyrrolidone (PVP-12), sodium caprylate, magnesium chloride, polysorbate 80, glyceryl monocaprylate, glyceryl ricaprylate, gelatin, gelatin capsules and Acryl-EZE® (methacrylate). The pharmaceutical compositions described herein include incorporation of octreotide as a therapeutic agent within an oral dosage form which is enteric-coated. An oral dosage form according to the invention comprises additives or excipients that are suitable for the preparation of the oral dosage form according to the present invention. The oral dosage form may comprise tablets or capsules, preferably enteric-coated. The administering of the oral octreotide may be once or twice a day in the morning and/or evening and occurs at least 1 hour before a meal or at least 2 hours after a meal.

In particular aspects of this invention the administration of oral octreotide comprises about 5 mg to about 400 mg of octreotide daily, about 10 to about 300 mg of octreotide daily, or about 40 to about 200 mg of octreotide daily, such as 5, 10, 20, 30, 40, 50, 60, 70, 80, 90, 100, 200, 300 or 400 mg daily. In an embodiment, oral octreotide is administered at 80-100 mg daily, for example twice daily for a total amount of 80-100 mg. The daily dose of octreotide may be administered in one or two doses a day e.g. the 80 mg daily dose may be administered in two doses of 40 mg each and a 100 mg dose may be administered twice daily each administration at 40 or 50 mg for example a 40 mg dose may be two 20 mg tablets.

One embodiment of the invention is a method of treatment of a subject suffering from a polycystic disease (such as polycystic kidney disease or polycystic liver disease or polycystic ovarian syndrome) or hypotension (in particular neurogenic orthostatic hypotension and postprandial hypotension) or diarrhea which comprises oral administration to the subject of a therapeutically effective amount of an oral somatostatin receptor ligand (SRL); in a particular embodiment the oral somatostatin receptor ligand (SRL) is an oral formulation of octreotide or lanreotide or pasireotide, or DG3173 preferably octreotide. In particular embodiments, the polycystic kidney disease is autosomal dominant polycystic kidney disease (ADPKD), the polycystic kidney disease is autosomal recessive polycystic kidney disease (ARPKD, the polycystic liver disease is a manifestation of autosomal dominant polycystic kidney disease (ADPKD) and the polycystic liver disease is autosomal dominant polycystic liver disease (ADPLD). In particular embodiments the diarrhea is intractable diarrhea, also termed refractory diarrhea. In particular embodiments the diarrhea is secretory diarrhea and the secretory diarrhea is chronic. In certain embodiments the diarrhea is caused by dumping syndrome, or by short bowel syndrome, by chemotherapy, by radiotherapy, by HIV/AIDS or by a neuroendocrine tumor (e.g. a carcinoid tumor or a Vasoactive Intestinal Peptide (VIP) secreting adenoma) or due to graft-versus-host disease, irritable bowel syndrome (IBS), inflammatory bowel disease (which includes conditions that cause the gut to become inflamed, such as Crohn's disease and ulcerative colitis), coeliac disease (also termed celiac sprue), chronic pancreatitis, diverticular disease, endocrine disorders, vasculitis, post-surgical diarrhea, carbohydrate malabsorption syndrome, amyloidosis, lactose intolerance, small bowel bacterial overgrowth, hepatobiliary disorders, inadequate luminal bile acid, bile acid malabsorption, loss of regulated gastric emptying, pancreatic exocrine insufficiency or neoplasia e.g. bowel cancer or may be due to be due to invasive infectious disease and/or bacterial endotoxins e.g. cholera.

Particular embodiments of the invention are a method of treatment wherein the oral administration of octreotide comprises about 5 mg to about 400 mg of octreotide daily or about 10 to about 300 mg of octreotide daily or about 40 to about 200 mg of octreotide daily or the administration of octreotide comprises about 5, 10, 20, 30, 40, 50, 60, 70, 80, 90, 100, 200, 300 or 400 mg daily, or the administration of octreotide comprises up to about 200 mg of octreotide daily.

In particular embodiments of the invention the oral administration of octreotide is once or twice per day in the morning and/or evening, and/or occurs at least 1 hour before a meal or at least 2 hours after a meal.

Another embodiment of the invention is a method of treatment of a subject suffering from a polycystic disease (such as polycystic kidney disease or polycystic liver disease or polycystic ovarian syndrome) which comprises oral administration to the subject of a therapeutically effective amount of an oral somatostatin receptor ligand (SRL) in combination with a therapeutically effective amount of a second or third therapeutic agent selected from the group consisting of an angiotensin-converting enzyme inhibitor, an angiotensin receptor blocker, an arginine vasopressin V2 receptor antagonist, a statin, a Src kinase inhibitor, and an mTOR inhibitor, or an injectable somatostatin receptor ligand (SRL).

In particular embodiments the oral somatostatin receptor ligand (SRL) is octreotide or lanreotide or pasireotide, preferably octreotide.

In particular embodiments of the combination therapy the polycystic kidney disease is autosomal dominant polycystic kidney disease (ADPKD), the polycystic kidney disease is autosomal recessive polycystic kidney disease (ARPKD), the polycystic liver disease is a manifestation of autosomal dominant polycystic kidney disease (ADPKD) or the polycystic liver disease is autosomal dominant polycystic liver disease (ADPLD).

In particular embodiments of the combination therapy the administration of octreotide comprises about 5 mg to about 400 mg of octreotide daily, about 10 to about 300 mg of octreotide daily or about 40 to about 200 mg of octreotide daily or administration of octreotide comprises about 5, 10, 20, 30, 40, 50, 60, 70, 80, 90, 100, 200, 300 or 400 mg daily, or up to about 200 mg of octreotide daily.

In particular embodiments of the combination therapy the administration of octreotide is once or twice per day in the morning and/or evening, and/or the administration of octreotide occurs at least 1 hour before a meal or at least 2 hours after a meal.

In particular embodiments of the combination therapy the angiotensin-converting enzyme inhibitor is lisinopril, the angiotensin receptor blocker inhibitor is telmisartan, the arginine vasopressin V2 receptor antagonist is tolvaptan, the statin is pravastatin, the Src kinase inhibitor is bosutinib and the mTOR inhibitor is everolimus or sirolimus.

One aspect of this invention is a method for treating a subject suffering from hypotension (e.g. neurogenic orthostatic hypotension or post prandial hypotension), the method comprising administration to the subject of a therapeutically effective amount of oral octreotide.

Another aspect of this invention is a method for treating a subject suffering from hypotension (e.g. neurogenic orthostatic hypotension or post prandial hypotension), the method comprising administration to the subject of a therapeutically effective amount of oral octreotide in combination with another drug used for treatment of neurogenic orthostatic hypotension or post prandial hypotension. For example, the drugs to be used in combination with oral octreotide include mineralocorticoids including but not limited to fludrocortisone. In some embodiments, oral octreotide is administered in combination with a large volume of physiological liquid (e.g., 100 mL, 200 mL, 500 mL or greater volume of physiological liquid). Thus an aspect of the invention is a method of treating a subject suffering from neurogenic orthostatic hypotension or post prandial hypotension, the method comprising administration to the subject of a therapeutically effective amount of an oral octreotide in combination with a high volume of water and a therapeutically effective amount of fludrocortisone or another antidiuretic agent.

In some embodiments, the methods described herein treat a subject suffering from hypotension (e.g., neurogenic orthostatic hypotension), the method comprising administering one or two doses (e.g., a dose comprising 1 to 2 tablets or capsules comprising octreotide) 15, 20, 30, 40, 45, or 60 minutes before getting up (e.g., in the morning or afternoon; before 4 or 5 pm), for example daily.

In some embodiments, the methods described herein treat a subject suffering from postprandial hypotension, the method comprising administering one or two doses (e.g., a dose comprising 1 to 2 tablets or capsules comprising octreotide) 15, 20, 30, 40, 45, or 60 minutes before a meal.

In some embodiments, the tablet or capsule comprising octreotide is about 10 to about 30 mg (e.g., about 15 to about 25 mg, about 18 to about 22 mg, about 20 mg) octreotide.

Another embodiment of the invention is a method of treatment of a subject suffering from diarrhea which comprises administration to the subject of a therapeutically effective amount of an oral somatostatin receptor ligand (SRL) in combination with a therapeutically effective amount of a second or third therapeutic agent selected from the group consisting of anti-diarrheal therapeutic agents, L-glutamine, teduglutide and injectable SRL. In particular embodiments the oral somatostatin receptor ligand (SRL) is octreotide or lanreotide or pasireotide, preferably octreotide. In particular embodiment of the combination therapy for diarrhea, the diarrhea is intractable diarrhea; in another embodiment the diarrhea is secretory diarrhea, which may be chronic. In certain embodiments of the invention the diarrhea is caused by dumping syndrome, or by short bowel syndrome, by chemotherapy, by radiotherapy, by HIV/AIDS or by a neuroendocrine tumor (e.g. a carcinoid tumor or a Vasoactive Intestinal Peptide (VIP) secreting adenoma) or due to graft-versus-host disease, irritable bowel syndrome (IBS), inflammatory bowel disease (which includes conditions that cause the gut to become inflamed, such as Crohn's disease and ulcerative colitis), coeliac disease (also termed celiac sprue), chronic pancreatitis, diverticular disease, endocrine disorders, vasculitis, post-surgical diarrhea, carbohydrate malabsorption syndrome, amyloidosis, lactose intolerance, small bowel bacterial overgrowth, hepatobiliary disorders, inadequate luminal bile acid, bile acid malabsorption, loss of regulated gastric emptying, pancreatic exocrine insufficiency or neoplasia e.g. bowel cancer or may be due to be due to invasive infectious disease and/or bacterial endotoxins e.g. cholera; in particular embodiments the diarrhea is caused by dumping syndrome, or by short bowel syndrome, by chemotherapy, by radiotherapy, by HIV/AIDS or by a neuroendocrine tumor (e.g. a carcinoid tumor).

In certain embodiments of the combination therapy for diarrhea the administration of octreotide comprises about 5 mg to about 400 mg of octreotide daily, or about 10 to about 300 mg of octreotide daily or about 40 to about 200 mg of octreotide daily or about 5, 10, 20, 30, 40, 50, 60, 70, 80, 90, 100, 200, 300 or 400 mg daily or up to about 200 mg of octreotide daily.

In certain embodiments of the combination therapy for diarrhea the administration of octreotide is once or twice per day in the morning and/or evening, and/or the administration of octreotide occurs at least 1 hour before a meal or at least 2 hours after a meal.

In certain embodiments of the combination therapy for diarrhea the anti-diarrheal therapeutic agents are selected from the group consisting of loperamide, cholestyramine, atropine, an opioid (e.g. codeine, diphenoxylate or difenoxin) and diphenoxylate/atropine (Lomotil®).

In certain embodiments of the combination therapy for diarrhea the second or third therapeutic agent is teduglutide or L-glutamine or an antibiotic. In certain embodiments of the combination therapy for diarrhea the diarrhea is caused by short bowel syndrome (SBS). In certain embodiments of the combination therapy for diarrhea the second or third therapeutic agent is selected from teduglutide, L-glutamine, histamine2-receptor agonists (H2 blockers), proton pump inhibitors (PPIs), clonidine, adsorbents/antisecretory agents (e.g. attapulgite, bismuth subsalicyclate, kaolin, pectin, crofelemer), bile acid sequestrants (binders) and antibiotics. In a particular embodiment the diarrhea is caused by short bowel syndrome (SBS).

Another embodiment of the invention is a unit dosage formulation for oral administration which comprises a therapeutically effective amount of an oral somatostatin receptor ligand (SRL) in combination with a therapeutically effective amount of a second or third therapeutic agent selected from the group consisting of an angiotensin-converting enzyme inhibitor, an angiotensin receptor blocker, an arginine vasopressin V2 receptor antagonist, a statin, a Src kinase inhibitor and an mTOR inhibitor. In a particular embodiment the oral SRL is octreotide or lanreotide or pasireotide, preferably octreotide. A particular embodiment of the unit dosage formulation comprises 5-120 mg octreotide. In particular embodiment of the unit dosage formulation the angiotensin-converting enzyme inhibitor is lisinopril, the angiotensin receptor blocker inhibitor is telmisartan, the arginine vasopressin V2 receptor antagonist is tolvaptan, the statin is pravastatin, the Src kinase inhibitor is bosutinib or the mTOR inhibitor is everolimus or sirolimus. In particular embodiments the unit dosage formulation comprises a tablet or a capsule. In particular embodiments the unit dosage formulation is for treatment of a subject suffering from a polycystic disease such as polycystic kidney disease or polycystic liver disease or polycystic ovarian syndrome.

Another aspect of this invention is a unit dosage formulation for oral administration comprising octreotide and a second oral drug used in treatment of neurogenic orthostatic hypotension or post prandial hypotension, including mineralocorticoids including but not limited to fludrocortisone.

Thus an embodiment of the invention is a unit dosage formulation for oral administration comprising octreotide and a second oral drug comprising a mineralocorticoid e.g. fludrocortisone. In some embodiments, the unit dosage formulation is administered in combination with a large volum of physiological liquid (e.g., 100 mL, 200 mL, 500 mL or greater volume of physiological liquid). In particular embodiments this unit dosage formulation may be used for treating neurogenic orthostatic hypotension and/or for treating post prandial hypotension.

Another embodiment of the invention is a unit dosage formulation which comprises a therapeutically effective amount of an oral somatostatin receptor ligand (SRL) in combination with a therapeutically effective amount of a second or third therapeutic agent selected from the group consisting of anti-diarrheal therapeutic agents, teduglutide, L-glutamine, histamine2-receptor agonists (H2 blockers), proton pump inhibitors (PPIs) and clonidine, adsorbents/antisecretory agents (e.g. attapulgite, bismuth subsalicyclate, kaolin, pectin, crofelemer), bile acid sequestrants (binders) and antibiotics.

In particular embodiments the oral SRL is octreotide or lanreotide or pasireotide, preferably octreotide. In particular embodiments the unit dosage formulation comprises about 5-120 mg octreotide, preferably about 10-40 mg octreotide. In particular embodiments of the unit dosage formulation the anti-diarrheal therapeutic agents are selected from the group consisting of loperamide, cholestyramine, atropine, an opioid (e.g. codeine, diphenoxylate or difenoxin) and diphenoxylate/atropine. In particular embodiments the unit dosage formulation comprises a tablet or a capsule. In particular embodiments the unit dosage formulation is for treatment of a subject suffering from diarrhea.

In particular embodiments the unit dosage combination formulation is for diarrhea caused by dumping syndrome, or by short bowel syndrome, by chemotherapy, by radiotherapy, by HIV/AIDS or by a neuroendocrine tumor (e.g. a carcinoid tumor or a Vasoactive Intestinal Peptide (VIP) secreting adenoma) or due to graft-versus-host disease, irritable bowel syndrome (IBS), inflammatory bowel disease (which includes conditions that cause the gut to become inflamed, such as Crohn's disease and ulcerative colitis), coeliac disease (also termed celiac sprue), chronic pancreatitis, diverticular disease, endocrine disorders, vasculitis, post-surgical diarrhea, carbohydrate malabsorption syndrome, amyloidosis, lactose intolerance, small bowel bacterial overgrowth, hepatobiliary disorders, inadequate luminal bile acid, bile acid malabsorption, loss of regulated gastric emptying, pancreatic exocrine insufficiency or neoplasia e.g. bowel cancer or may be due to be due to invasive infectious disease and/or bacterial endotoxins e.g. cholera. In particular embodiments the unit dosage combination formulation is for diarrhea caused by dumping syndrome, or by short bowel syndrome, by chemotherapy, by radiotherapy, by HIV/AIDS or by a neuroendocrine tumor (e.g. a carcinoid tumor).

One embodiment of the invention is a method of treatment of a subject suffering from a neuroendocrine tumor which comprises administration to the subject of a therapeutically effective amount of an oral somatostatin receptor ligand (SRL) in combination with a therapeutically effective amount of one or more anti-tumor agents or an mTOR inhibitor or an VEGFR inhibitor, or an Src kinase inhibitor or a tryptophan hydroxylase inhibitor, or an injectable somatostatin receptor ligand (SRL). In one embodiment the method of treatment comprises administering a therapeutically effective amount of a third therapeutic agent.

In a particular embodiment, the oral somatostatin receptor ligand (SRL) is and oral formulation of octreotide, lanreotide, pasireotide or DG3173, preferably octreotide. In a particular embodiment the injectable somatostatin receptor ligand is a long-acting injectable formulation; in another particular embodiment of the invention the injectable somatostatin receptor ligand is octreotide, lanreotide, pasireotide, DG3173 or CAM2029.

In a particular embodiment the mTOR inhibitor is everolimus, temsirolimus or sirolimus. In a particular embodiment of the invention the VEGFR inhibitor is sunitinib. In a particular embodiment of the invention the Src kinase inhibitor is bosutinib. In a particular embodiment of the invention the tryptophan hydroxylase inhibitor is telotristat etiprate.

In particular embodiments the anti-tumor agent is selected from alkylating agents, doxorubicin, 5-fluorouracil, dacarbazine, actinomycin D, platinum compounds (cisplatin, carboplatin, oxaliplatin), irinotecan, etoposide, streptozotocin, interferon alfa, interferon gamma, bortezomib, temozolomide, bevacizumab, capecitabine and somatostatin analogs with a radioactive load or a combination thereof.

In certain embodiments, administration of octreotide comprises about 5 mg to about 400 mg of octreotide daily, or about 40 to about 200 mg of octreotide daily or about 10 to about 120 mg of octreotide daily, such as 10, 20, 30, 40, 50, 60, 70, 80, 90 or 100, 110 or 120 mg or 200 mg or 300 mg or 400 mg or more daily.

In certain embodiments, the long-acting injectable formulation is administered every three, four, five, or six weeks preferably every four weeks. In particular embodiments the administration of oral octreotide is in order to treat breakthrough neuroendocrine tumor symptoms, such as diarrhea, facial flushing and abdominal pain, and in certain embodiments of the invention the octreotide is administered on an “on demand” basis.

The success of the treatment may be measured by a reduction in the average number of daily bowel movements of a subject suffering from diarrhea due to NET after some weeks of treatment e.g. 6-12 weeks compared with baseline.

Another embodiment of the invention is a unit dosage formulation for oral administration comprising an oral SRL and a second therapeutic agent; this may be for treatment of a neuroendocrine tumor. In particular embodiments the oral SRL is an oral formulation of octreotide or lanreotide or pasireotide or DG3173 (also termed somatoprim, a novel SRL); in a particular embodiment the SRL is octreotide. In certain embodiments the second therapeutic agent is an oral anti-tumor agent or an oral mTOR inhibitor or an oral VEGFR inhibitor or an oral Src kinase inhibitor. In certain embodiments the oral anti-tumor agent is selected from an oral form of alkylating agents, doxorubicin, fluorpyrimidines e.g. 5-fluorouracil, dacarbazine, actinomycin D, platinum compounds (cisplatin, carboplatin, oxaliplatin), irinotecan, etoposide, streptozotocin, temozolomide, bevacizumab and capecitabine. In certain embodiments the oral mTOR inhibitor is everolimus or sirolimus. In certain embodiments the oral VEGFR inhibitor is sunitinib. In certain embodiments the oral Src kinase inhibitor is bosutinib. In certain embodiments the oral tryptophan hydroxylase inhibitor is telotristat etiprate; this unit dosage formulation may be for a subject suffering from a neuroendocrine tumor or from acromegaly. In particular embodiments the unit dosage formulation comprises 5-400 mg octreotide. In certain embodiments the unit dosage formulation additionally comprises a therapeutically effective amount of a third therapeutic agent.

One measure of the success of the treatment (of a subject suffering from diarrhea) comprising oral octreotide alone or in combination with a second or third therapeutic anti-diarrheal agent, is a reduction in the average number of daily bowel movements of the subject suffering from diarrhea after some weeks of treatment e.g. 6-12 weeks compared with baseline. Another measure of success is reduction in volume of daily bowel movements of the subject suffering from diarrhea after some weeks of treatment e.g. 6-12 weeks compared with baseline

Administered “in combination”, as used herein, means that two (or more) different therapeutic agents are delivered to the subject during the course of the subject's affliction with the disorder, e.g., the two or more therapeutic agents are delivered after the subject has been diagnosed with the disorder and before the disorder has been cured or eliminated or treatment has ceased for other reasons. In some embodiments, the delivery of one therapeutic agent is still occurring when the delivery of the second begins, so that there is overlap in terms of administration. This is sometimes referred to herein as “simultaneous” or “concurrent delivery”. In other embodiments, the delivery of one therapeutic agent ends before the delivery of the other treatment begins. In some embodiments of either case, the therapeutic agents are more effective because of combined administration. For example, the second therapeutic agent is more effective, e.g., an equivalent effect is seen with less of the second therapeutic agent, or the second therapeutic agent reduces symptoms to a greater extent, than would be seen if the second therapeutic agent were administered in the absence of the first therapeutic agent, or the analogous situation is seen with the first therapeutic agent. In some embodiments, delivery is such that the reduction in a symptom, or other parameter related to the disorder is greater than what would be observed with one therapeutic agent delivered in the absence of the other. The effect of the two therapeutic agents can be partially additive, wholly additive, or greater than additive. The delivery can be such that an effect of the first therapeutic agent delivered is still detectable when the second is delivered.

The compositions described herein can be administered to a subject i.e., a human or an animal, in order to treat the subject with a pharmacologically or therapeutically effective amount of a therapeutic agent described herein. The animal may be a mammal e.g., a mouse, rat, pig, dog horse, cow or sheep. As used herein the terms “pharmacologically effective amount” or “therapeutically effective amount” or “effective amount” means that amount of a drug or pharmaceutical agent (the therapeutic agent) that will elicit the biological or medical response of a tissue, system, animal or human that is being sought by a researcher or clinician and/or halts or reduces the progress of the condition being treated or which otherwise completely or partly cures or acts palliatively on the condition, or prevents development of the condition.

As used herein, the term “treatment” as for example in “method of treatment” or “treat” or “treating” refers to therapeutic treatment, wherein the object is to reduce or reverse or prevent the symptoms of a disease or disorder. In some embodiments, the compounds or compositions disclosed herein are administered prior to onset of the disease or disorder. In some embodiments, the compounds or compositions disclosed herein are during or subsequent to the onset of the disease or disorder.

The function and advantages of these and other embodiments will be more fully understood from the following examples. These examples are intended to be illustrative in nature and are not to be considered as limiting the scope of the systems and methods discussed herein.

### EXAMPLES

### Example 1: Clinical Experiment on Oral Octreotide for Treatment of Polycystic Kidney Disease

Patients with autosomal polycystic kidney disease are randomly assigned to be treated with oral octreotide (for example up to 100 mg daily administered twice daily) or placebo, in addition to standard of care.

### Primary Outcome Measures:

Change over baseline of the total kidney volume at one-year follow-up (estimated by gadolinium contrast enhanced and T2-weighted magnetic resonance imaging, MRI). and/or
The slope through serial estimated glomerular filtration rate (eGFR) measurements starting at week 12 until end of treatment.

### Secondary Outcome Measures:

Absolute and percent change over baseline by MRI analysis are compared in the two ADPKD groups at baseline, at one-year follow-up
Total renal parenchymal volume (basal and one-year follow up)
Residual renal volume (basal and one-year follow up)
Renal parenchymal volume taken up by small cysts, minor of five mm cubic (basal and one-year follow up)

### Example 2: Clinical Experiment on Oral Octreotide for Treatment of Polycystic Liver Disease

Patients with autosomal polycystic liver disease are randomly assigned to be treated with oral octreotide (for example up to 100 mg daily administered twice daily) or placebo, in addition to standard of care.

### Primary Outcome Measures:

Percent change in liver volume (basal and one-year follow up)
Percent change from baseline in liver volume, measured in milliliters by Magnetic Resonance Imaging (MRI) or Computed Tomography (CT) scans

### Secondary Outcome Measures:

Percent change in renal volume (basal and one-year follow up)
Percent change from baseline in renal volume, measured in milliliters by MRI or CT scans

### Example 3: Clinical Experiment on Oral Octreotide in Combination with Another Drug for Treatment of Polycystic Kidney or Liver Disease

Patients with autosomal polycystic kidney or liver disease are randomly assigned to be treated with oral octreotide alone (for example up to 100 mg daily administered twice daily) or placebo, or the combination (octreotide plus 2nd therapeutic agent) or the 2nd therapeutic agent alone, all in addition to standard of care. The 2nd therapeutic agent is selected from: lisinopril, telmisartan, tolvaptan, pravastatin, bosutinib, everolimus and sirolimus.

The primary and secondary outcome measures are as described above.

### Example 4: Clinical Experiment on Oral Octreotide for Treatment of Diarrhea

Patients with severe diarrhea (e.g. having short bowel syndrome) are randomly assigned to be treated with oral octreotide (for example up to 80 mg daily administered twice daily) in combination with an antidiarrheal agent or placebo. The number of daily stools (bowel movements) and their volume are determined at start of study (baseline) and every 4 weeks thereafter. The study continues for 24 weeks, with measurement every 4 weeks.

Primary Outcome Measures:
1. Reduction in number of daily stools (bowel movements) compared with baseline
2. Reduction in volume of daily stools compared with baseline

Secondary Outcome Measure:

Reduction in IV nutrition volume compared to baseline

Having thus described several aspects of at least one embodiment, it is to be appreciated that various alterations, modifications, and improvements will readily occur to those skilled in the art. Such alterations, modifications, and improvements are intended to be part of this disclosure and are intended to be within the scope of the invention. Accordingly, the foregoing description is by way of example only, and the scope of the invention should be determined from proper construction of the appended claims, and their equivalents.

1. A method of treatment of a subject suffering from polycystic disease or hypotension or diarrhea which comprises oral administration to the subject of a therapeutically effective amount of an oral somatostatin receptor ligand (SRL).

2. The method of treatment of claim 1 wherein the oral somatostatin receptor ligand (SRL) is an oral formulation of octreotide or lanreotide or pasireotide or DG3173.

3. The method of treatment of claims 1-2 wherein the oral somatostatin receptor ligand (SRL) is octreotide.

4. The method of claims 1-3 wherein the polycystic disease is polycystic kidney disease or polycystic liver disease or polycystic ovarian syndrome.

5. The method of claim 4 wherein the polycystic kidney disease is autosomal dominant polycystic kidney disease (ADPKD).

6. The method of claim 4 wherein the polycystic kidney disease is autosomal recessive polycystic kidney disease (ARPKD).

7. The method of claim 4 wherein the polycystic liver disease is a manifestation of autosomal dominant polycystic kidney disease (ADPKD) or is autosomal dominant polycystic liver disease (ADPLD).

8. The method of treatment of claims 1-3 wherein the diarrhea is intractable diarrhea.

9. The method of treatment of claims 1-3 wherein the diarrhea is secretory diarrhea.

10. The method of treatment of claims 1-3 wherein the secretory diarrhea is chronic.

11. The method of treatment of claims 1-3 wherein the diarrhea is caused by dumping syndrome, or by short bowel syndrome, by chemotherapy, by radiotherapy, by HIV/AIDS or by a neuroendocrine tumor (e.g. a carcinoid tumor or a Vasoactive intestinal Peptide (VIP) secreting adenoma) or due to graft-versus-host disease, irritable bowel syndrome (IBS), inflammatory bowel disease (which includes conditions that cause the gut to become inflamed, such as Crohn's disease and ulcerative colitis), coeliac disease (also termed celiac sprue), chronic pancreatitis, diverticular disease, endocrine disorders, vasculitis, post-surgical diarrhea, carbohydrate malabsorption syndrome, amyloidosis, lactose intolerance, small bowel bacterial overgrowth, hepatobiliary disorders, inadequate luminal bile acid, bile acid malabsorption, loss of regulated gastric emptying, pancreatic exocrine insufficiency or neoplasia e.g. bowel cancer or may be due to be due to invasive infectious disease and/or bacterial endotoxins e.g. cholera.

12. The method of claims 3-11 wherein the administration of octreotide comprises about 5 mg to about 400 mg of octreotide daily.

13. The method of claims 3-11 wherein the administration of octreotide comprises about 10 to about 300 mg of octreotide daily.

14. The method of claims 3-11 wherein the administration of octreotide comprises about 40 to about 200 mg of octreotide daily.

15. The method of claim 12 wherein the administration of octreotide comprises about 5, 10, 20, 30, 40, 50, 60, 70, 80, 90, 100, 200, 300 or 400 mg daily.

16. The method of claim 15 wherein the administration of octreotide comprises up to 200 mg of octreotide daily.

17. The method of claims 3-16 wherein the administration of octreotide is once or twice per day in the morning and/or evening.

18. The method of claims 3-17 wherein the administration of octreotide occurs at least 1 hour before a meal or at least 2 hours after a meal.

19. The method of claims 1-3 and 12-18 where the hypotension is orthostatic hypotension, in particular neurogenic orthostatic hypotension.

20. The method of claims 1-3 and 12-18 where the hypotension is postprandial hypotension.

21. A method of treatment of a subject suffering from a polycystic disease which comprises oral administration to the subject of a therapeutically effective amount of an oral somatostatin receptor ligand (SRL) in combination with a therapeutically effective amount of a second or third therapeutic agent selected from the group consisting of an angiotensin-converting enzyme inhibitor, an angiotensin receptor blocker, an arginine vasopressin V2 receptor antagonist, a statin, a Src kinase inhibitor, an mTOR inhibitor and an injectable somatostatin receptor ligand (SRL).

22. The method of treatment of claim 21 where the polycystic disease is polycystic kidney disease or polycystic liver disease or polycystic ovarian syndrome.

23. The method of treatment of claim 21 wherein the oral somatostatin receptor ligand (SRL) is an oral formulation of octreotide or lanreotide or pasireotide or DG3173.

24. The method of treatment of claim 23 wherein the oral somatostatin receptor ligand (SRL) is octreotide.

25. The method of treatment of claims 21-23 wherein the injectable somatostatin receptor ligand is a long-acting injectable formulation.

26. The method of treatment of claim 25 wherein the injectable somatostatin receptor ligand is octreotide, lanreotide, pasireotide, DG3173 or CAM2029.

27. The method of claims 22-26 wherein the polycystic kidney disease is autosomal dominant polycystic kidney disease (ADPKD).

28. The method of claims 22-26 wherein the polycystic kidney disease is autosomal recessive polycystic kidney disease (ARPKD).

29. The method of claims 22-26 wherein the polycystic liver disease is a manifestation of autosomal dominant polycystic kidney disease (ADPKD) or wherein the polycystic liver disease is autosomal dominant polycystic liver disease (ADPLD).

30. The method of claims 24-29 wherein the administration of octreotide comprises about 5 mg to about 400 mg of octreotide daily.

31. The method of claim 30 wherein the administration of octreotide comprises about 10 to about 300 mg of octreotide daily.

32. The method of claim 30 wherein the administration of octreotide comprises about 40 to about 200 mg of octreotide daily.

33. The method of claim 30 wherein the administration of octreotide comprises about 5, 10, 20, 30, 40, 50, 60, 70, 80, 90, 100, 200, 300 or 400 mg daily.

34. The method of claim 33 wherein the administration of octreotide comprises up to about 200 mg of octreotide daily.

35. The method of claims 24-34 wherein the administration of octreotide is once or twice per day in the morning and/or evening.

36. The method of claims 24-35 wherein the administration of octreotide occurs at least 1 hour before a meal or at least 2 hours after a meal.

37. The method of claims 21-36 wherein the angiotensin-converting enzyme inhibitor is lisinopril.

38. The method of claim 21-36 wherein the angiotensin receptor blocker inhibitor is telmisartan.

39. The method of claim 21-36 wherein the arginine vasopressin V2 receptor antagonist is tolvaptan.

40. The method of claim 21-36 wherein the statin is pravastatin.

41. The method of claim 21-36 wherein the Src kinase inhibitor is bosutinib.

42. The method of claim 21-36 wherein the mTOR inhibitor is everolimus or sirolimus.

43. A method of treatment of a subject suffering from diarrhea which comprises oral administration to the subject of a therapeutically effective amount of an oral somatostatin receptor ligand (SRL) in combination with a therapeutically effective amount of a second or third therapeutic agent selected from the group consisting of anti-diarrheal therapeutic agents, teduglutide, L-glutamine and injectable SRL.

44. The method of treatment of claim 43 wherein the oral somatostatin receptor ligand (SRL) is an oral formulation of octreotide or lanreotide or pasireotide or DG3173.

45. The method of treatment of claim 43 wherein the oral somatostatin receptor ligand (SRL) is octreotide.

46. The method of treatment of claims 43-45 wherein the injectable somatostatin receptor ligand is a long-acting injectable formulation.

47. The method of treatment of claim 46 wherein the injectable somatostatin receptor ligand is octreotide, lanreotide, pasireotide, DG3173 or CAM2029.

48. The method of treatment of claims 43-47 wherein the diarrhea is intractable diarrhea.

49. The method of treatment of claims 43-47 wherein the diarrhea is secretory diarrhea.

50. The method of treatment of claim 49 wherein the secretory diarrhea is chronic.

51. The method of treatment of claims 39-41 wherein the diarrhea is caused by dumping syndrome, or by short bowel syndrome (SBS), by chemotherapy, by radiotherapy, by HIV/AIDS or by a neuroendocrine tumor (e.g. a carcinoid tumor or a Vasoactive Intestinal Peptide (VIP) secreting adenoma) or due to graft-versus-host disease, irritable bowel syndrome (IBS), inflammatory bowel disease (which includes conditions that cause the gut to become inflamed, such as Crohn's disease and ulcerative colitis), coeliac disease (also termed celiac sprue), chronic pancreatitis, diverticular disease, endocrine disorders, vasculitis, post-surgical diarrhea, carbohydrate malabsorption syndrome, amyloidosis, lactose intolerance, small bowel bacterial overgrowth, hepatobiliary disorders, inadequate luminal bile acid, bile acid malabsorption, loss of regulated gastric emptying, pancreatic exocrine insufficiency or neoplasia e.g. bowel cancer or may be due to be due to invasive infectious disease and/or bacterial endotoxins e.g. cholera.

52. The method of claims 43-51 wherein the administration of octreotide comprises about 5 mg to about 400 mg of octreotide daily.

53. The method of claims 43-51 wherein the administration of octreotide comprises about 10 to about 300 mg of octreotide daily.

54. The method of claims 43-51 wherein the administration of octreotide comprises about 40 to about 200 mg of octreotide daily.

55. The method of claim 52 wherein the administration of octreotide comprises about 5, 10, 20, 30, 40, 50, 60, 70, 80, 90, 100, 200, 300 or 400 mg daily.

56. The method of claim 55 wherein the administration of octreotide comprises up to about 200 mg of octreotide daily.

57. The method of claims 43-56 wherein the administration of octreotide is once or twice per day in the morning and/or evening.

58. The method of claims 43-57 wherein the administration of octreotide occurs at least 1 hour before a meal or at least 2 hours after a meal.

59. The method of claims 39-58 wherein the anti-diarrheal therapeutic agents are selected from the group consisting of loperamide, cholestyramine, atropine and an opioid (e.g. codeine, diphenoxylate or difenoxin).

60. The method of claims 39-58 wherein the second or third therapeutic agent is teduglutide or L-glutamine and wherein the diarrhea is caused by short bowel syndrome (SBS).

61. A unit dosage formulation for oral administration which comprises a therapeutically effective amount of an oral somatostatin receptor ligand (SRL) in combination with a therapeutically effective amount of a second or third therapeutic agent selected from the group consisting of an angiotensin-converting enzyme inhibitor, an angiotensin receptor blocker, an arginine vasopressin V2 receptor antagonist, a statin, a Src kinase inhibitor and an mTOR inhibitor.

62. The unit dosage formulation of claim 61 wherein the oral SRL is an oral formulation of octreotide or lanreotide or pasireotide or DG3173, preferably octreotide.

63. The unit dosage formulation of claim 62 which comprises 5-200 mg octreotide.

64. The unit dosage formulation of claim 61 wherein the angiotensin-converting enzyme inhibitor is lisinopril.

65. The unit dosage formulation of claim 61 wherein the angiotensin receptor blocker inhibitor is telmisartan.

66. The unit dosage formulation of claim 61 wherein the arginine vasopressin V2 receptor antagonist is tolvaptan.

67. The unit dosage formulation of claim 61 wherein the statin is pravastatin.

68. The unit dosage formulation of claim 61 wherein the Src kinase inhibitor is bosutinib.

69. The unit dosage formulation of claim 61 wherein the mTOR inhibitor is everolimus or sirolimus.

70. The unit dosage formulation of claims 61-69 which comprises a tablet or a capsule.

71. The unit dosage formulation of claims 61-70 for treatment of a subject suffering from polycystic kidney disease or polycystic liver disease or polycystic ovarian syndrome.

72. A unit dosage formulation for oral administration which comprises a therapeutically effective amount of an oral somatostatin receptor ligand (SRL) in combination with a therapeutically effective amount of a second or third therapeutic agent selected from the group consisting of anti-diarrheal therapeutic agents, L-glutamine and teduglutide.

73. The unit dosage formulation of claim 72 wherein the oral SRL is an oral formulation of octreotide or lanreotide or pasireotide or DG3173, preferably octreotide.

74. The unit dosage formulation of claim 73 which comprises about 5-120 mg octreotide, preferably about 10-40 mg octreotide.

75. The unit dosage formulation of claims 72-74 wherein the anti-diarrheal therapeutic agents are selected from the group consisting of loperamide, cholestyramine, atropine, an opioid (e.g. codeine, diphenoxylate or difenoxin).

76. The unit dosage formulation of claims 72-75 which comprises a tablet or a capsule.

77. The unit dosage formulation of claims 72-76 for treatment of a subject suffering from diarrhea.

78. The unit dosage formulation of claim 77 wherein the diarrhea is caused by dumping syndrome, or by short bowel syndrome, by chemotherapy, by radiotherapy, by HIV/AIDS or by a neuroendocrine tumor (e.g. a carcinoid tumor or a Vasoactive Intestinal Peptide (VIP) secreting adenoma) or due to graft-versus-host disease, irritable bowel syndrome (IBS), inflammatory bowel disease (which includes conditions that cause the gut to become inflamed, such as Crohn's disease and ulcerative colitis), coeliac disease (also termed celiac sprue), chronic pancreatitis, diverticular disease, endocrine disorders, vasculitis, post-surgical diarrhea, carbohydrate malabsorption syndrome, amyloidosis, lactose intolerance, small bowel bacterial overgrowth, hepatobiliary disorders, inadequate luminal bile acid, bile acid malabsorption, loss of regulated gastric emptying, pancreatic exocrine insufficiency or neoplasia e.g. bowel cancer or may be due to be due to invasive infectious disease and/or bacterial endotoxins e.g. cholera.

79. The unit dosage formulation of claim 78 wherein the diarrhea is caused by dumping syndrome, or by short bowel syndrome, by chemotherapy, by radiotherapy, by HIV/AIDS or by a neuroendocrine tumor or carcinoid syndrome.

80. The unit dosage formulation of claim 79 wherein the diarrhea is caused by short bowel syndrome and wherein the second or third therapeutic agent is teduglutide or L-glutamine.

81. A method of treatment of a subject suffering from a neuroendocrine tumor or carcinoid syndrome which comprises oral administration to the subject of a therapeutically effective amount of an oral somatostatin receptor ligand (SRL) in combination with a therapeutically effective amount of one or more anti-tumor agents or an mTOR inhibitor or an VEGFR inhibitor or an Src kinase inhibitor or a tryptophan hydroxylase inhibitor or an injectable somatostatin receptor ligand (SRL).

82. The method of treatment of claim 81 wherein the oral somatostatin receptor ligand (SRL) is an oral formulation of octreotide, lanreotide, pasireotide, or DG3173, preferably octreotide.

83. The method of treatment of claims 81-82 wherein the injectable somatostatin receptor ligand is a long-acting injectable formulation.

84. The method of treatment of claim 83 wherein the injectable somatostatin receptor ligand is octreotide, lanreotide, pasireotide, DG3173 or CAM2029.

85. The method of claims 81-84 wherein the mTOR inhibitor is everolimus, temsirolimus or sirolimus.

86. The method of claims 81-84 wherein the VEGFR inhibitor is sunitinib.

87. The method of claims 81-84 wherein the Src kinase inhibitor is bosutinib.

88. The method of claims 81-84 wherein the tryptophan hydroxylase inhibitor is telotristat etiprate.

89. The method of claims 81-84 wherein the anti-tumor agent is selected from alkylating agents, doxorubicin, fluoropyrimidines e.g. 5-fluorouracil, dacarbazine, actinomycin D, platinum compounds (cisplatin, carboplatin, oxaliplatin), irinotecan, etoposide, streptozotocin, interferon alfa, interferon gamma, bortezomib, temozolomide, bevacizumab, capecitabine and somatostatin analogs with a radioactive load or a combination thereof.

90. The method of claims 81-89 wherein the administration of octreotide comprises about 5 mg to about 400 mg of octreotide daily.

91. The method of claim 90 wherein the administration of octreotide comprises about 40 to about 400 mg of octreotide daily.

92. The method of claim 90 wherein the administration of oral octreotide comprises about 10 to about 100 mg of octreotide daily, such as 10, 20, 30, 40, 50, 60, 70, 80, 90 or 100 or 200 mg or 300 mg or 400 mg or more daily.

93. The method of claims 24-25, 46-47 and 83-84 wherein the long-acting injectable formulation is administered every three, four, five, or six weeks preferably every four weeks.

94. The method of claim 93 wherein the administration of oral octreotide is in order to treat breakthrough symptoms.

95. The method of claims 94-95 wherein the oral octreotide is administered on an “on demand” basis.

96. The method of claim 94, wherein the breakthrough symptoms related to carcinoid syndrome are diarrhea, facial flushing and abdominal pain.

97. A unit dosage formulation for oral administration comprising an oral SRL and a second therapeutic agent.

98. The unit dosage formulation of claim 97 wherein the oral SRL is an oral formulation of octreotide or lanreotide or pasireotide or DG3173.

99. The unit dosage formulation of claim 98 wherein the oral SRL is octreotide.

100. The unit dosage formulation of claims 97-99 wherein the second therapeutic agent is an oral anti-tumor agent or an oral mTOR inhibitor or an oral VEGFR inhibitor or an oral Src kinase inhibitor or a tryptophan hydroxylase inhibitor.

101. The unit dosage formulation of claim 100 wherein the oral anti-tumor agent is selected from an oral form of alkylating agents, doxorubicin, 5-fluorouracil, dacarbazine, actinomycin D, platinum compounds (cisplatin, carboplatin, oxaliplatin), irinotecan, etoposide, streptozotocin, temozolomide, bevacizumab and capecitabine.

102. The unit dosage formulation of claim 100 wherein the oral mTOR inhibitor is everolimus or sirolimus.

103. The unit dosage formulation of claim 100 wherein the oral VEGFR inhibitor is sunitinib.

104. The unit dosage formulation of claim 100 wherein the oral Src kinase inhibitor is bosutinib.

105. The unit dosage formulation of claim 100 wherein the oral tryptophan hydroxylase inhibitor is telotristat etiprate.

106. The unit dosage formulation of claims 100-105 for treatment of a subject suffering from a neuroendocrine tumor.

107. The unit dosage formulation of claim 105 for treatment of a subject suffering from acromegaly.

108. The unit dosage formulation of claims 97-107 which comprises 5-200 mg octreotide.

109. The unit dosage formulation of claim 97 which additionally comprises a therapeutically effective amount of a third therapeutic agent.

110. The method of claims 81-96 which additionally comprises administering a therapeutically effective amount of a third therapeutic agent.

111. A method of treatment of a subject suffering from acromegaly which comprises oral administration to the subject of a therapeutically effective amount of an oral somatostatin receptor ligand (SRL) in combination with a therapeutically effective amount of a tryptophan hydroxylase inhibitor.

112. A method of treatment of claim 111 wherein the tryptophan hydroxylase inhibitor is telotristat etiprate.

113. A method of treating a subject suffering from neurogenic orthostatic hypotension or post prandial hypotension, the method comprising administration to the subject of a therapeutically effective amount of an oral octreotide in combination with a therapeutically effective amount of fludrocortisone or another antidiuretic agent.

114. A method of treating a subject suffering from neurogenic orthostatic hypotension or post prandial hypotension, the method comprising administration to the subject of a therapeutically effective amount of an oral octreotide in combination with a high volume of water and a therapeutically effective amount of fludrocortisone or another antidiuretic agent.

115. A unit dosage formulation for oral administration comprising a therapeutically effective amount of octreotide in combination with a therapeutically effective amount of fludrocortisone or another antidiuretic agent.

116. The unit dosage formulation of claim 115 for treating neurogenic orthostatic hypotension.

117. The unit dosage formulation of claim 115 for treating post prandial hypotension.
